(19) United States
(12) Patent Application Publication
(10) Pub. No.: US 2006/0106434 A1
(43) Pub. Date: May 18, 2006

(54) Portable time harmonic tissue stimulator and method

(76) Inventors: Steven T. Padgitt, Felton, CA (US); Michael D. McPherson, Santa Cruz, CA (US)

Correspondence Address: Steven T. Padgitt;Suite 200, 725 Front Street, Santa Cruz, CA, 95060, US

(21) Appl. No.: 10/986,363

(22) Filed: Nov. 12, 2004

(51) Int. Cl. A61N 1/32

(52) U.S. Cl. 607/66

(57) ABSTRACT

A programmable electronic time harmonic therapeutic and research device used in stimulating living human and other living tissue. This invention consists of a portable, electronic apparatus, multiple types of stimulus delivery, multiple methods of stimulus delivery, electromagnetic, electric and other manner of emitters, and multiple emitter housing apparatus. Electric and electromagnetic and other manner of stimulation may be used in a complex of ways for therapeutic and research purposes. Conditioning and learning methods, principles and techniques, are incorporated into settings for tissue stimulation sessions for the re-conditioning and re-learning of conditioned and maladaptive responses. This invention is, in part, designed for the entrainment of neurons as a part of the therapeutic effect. The size and portability of this device makes it suitable for patient and enthusiast home use, as well as practitioner office, and research facility. It is used for a multiplicity of purposes, including mental and physical health concerns, meditation, self-improvement, and a broad base of living tissue stimulation research.

### CROSS-REFERENCED TO RELATED APPLICATIONS

Provisional Patent Application No. 60/518,579 submitted on Nov. 7, 2003 and received by USPTO on Nov. 10, 2003

### FEDERALLY SPONSORED RESEARCH

Not Applicable

### SEQUENCE LISTING

Not Applicable

### REFERENCES CITED

### U.S. Patent Documents

| | | | |
|---|---|---|---|
| 435376 | August, 1890 | Brown | 600/13. |
| 3753433 | August, 1973 | Bakerich et al. | 600/545. |
| 3773049 | November, 1973 | Rabichev et al. | 600/28. |
| 4227516 | October, 1980 | Meland et al. | 600/26. |
| 4335710 | June, 1982 | Williamson | 600/28. |
| 4428366 | January, 1984 | Findl et al. | 600/14. |
| 4834701 | May, 1989 | Masaki | 600/28. |
| 4902274 | February, 1990 | Gleeson, III | 600/27. |
| 5002068 | March, 1991 | Powell | 128/846. |
| 5036858 | August, 1991 | Carter et al. | 600/27. |
| 5047005 | September, 1991 | Cadwell | 600/13. |
| 5108361 | April, 1992 | Hein | 600/28. |
| 5224922 | July, 1993 | Kurtz | 600/9. |
| 5306228 | April, 1994 | Rubins | 600/27. |
| 5356368 | October, 1994 | Monroe | 600/28. |
| 5470846 | November, 1995 | Sandyk | 519/159. |
| 5480374 | January, 1996 | Van Dick | 600/26. |
| 5518495 | May, 1996 | Kolt | 600/13. |
| 5527259 | June, 1996 | Grace et al. | 600/14. |
| 5788624 | August, 1998 | Lu et al. | 600/15. |
| 5885976 | March, 1999 | Sandyk | 519/159. |
| 5908444 | June, 1999 | Azure | 600/14. |
| 5935054 | Aug. 10, 1999 | Loos | 600/9 |
| 6071229 | June, 2000 | Rubins | 600/27. |
| 6128174 | October, 2000 | Ritter et al. | 600/13. |
| 6132361 | October, 2000 | Epstein, et al. | 600/13 |
| 6234953 | May, 2001 | Thomas et al. | 600/14. |
| 6402678 | June, 2002 | Fischell, et al. | 600/13 |
| 6425852 | July, 2002 | Epstein, et al. | 600/13 |
| 6447440 | September, 2002 | Markoll | 600/13 |
| 6450940 | September, 2002 | Moritz, et al. | 600/13 |
| 6461289 | October, 2002 | Muntermann | 600/9 |
| 6520903 | February, 2003 | Yamashiro | 600/9 |
| 6527696 | March, 2003 | Kim | 600/13 |
| 6527697 | March, 2003 | Bashford, et al. | 600/13 |
| 6572528 | Jun. 3, 2003 | Rohan, et al. | 600/14 |

### BACKGROUND OF INVENTION

1. Field of Invention

This invention relates to tissue stimulation, including electromagnetic stimulation of neuronal tissue, used for enhancing adaptive qualities and functioning of tissue.

2. Prior Art

This invention provides new, expanded and programmable forms of stimulation to living tissue, for application to human and other animal tissue, including the central and peripheral nervous systems and is intended and designed to promote enhanced and positive nervous system activity and performance in accordance with recent advances in scientific and academic research in the fields of psychiatry, psychology and human cognition.

This invention is intended to provide a unique contribution to the achievement of the health-related objectives of consumers and health and research practitioners by providing new technology and systematic protocols for the advancement of the user's self-regulation of internal emotional, psychological, biochemical and physiological states.

The invention provides for a systematic learning tool in the management of high stress and undesirable physical and emotional conditions through the deployment of different methods, strategies and devices that can be regulated by a health care practitioner or the end user. This invention incorporates recent advances in technology to deliver time harmonic or repetitive electromagnetic, electric and other manner of stimulation of tissue. The invention provides choices for the channeling of the stimulation through the cranium to the central nervous system, to the peripheral nervous system and/or to other muscle, bone or organ tissue of human or other animal bodies.

This patent application applies to a unique portable apparatus capable of delivering a plurality of tissue stimulation types, using electromagnetic, electric and other methods of stimulation via a multiplicity stimulation variables including sequential and or nonsequential frequencies, magnitudes, and phases.

This invention also applies to the sequencing and pattern of time harmonic delivery, within a plurality of output delivery ports. These ports are capable of simultaneous delivery of the programmed forms of stimulation. This device will use a natural central nervous system process, in which neurons and networks of neurons are known to recruit other neurons and networks of neurons.

This patent includes unique method, procedure and protocol the inventors refer to as, Time Harmonic Biodesensitization and Conditioning [hereinafter referred to as THBC]. THBC combines time harmonic stimulation, including repetitive Transcranial Magnetic Stimulation [hereafter TMS], with classical and operant conditioning procedures, processes, protocols and outcomes, as well as other methods of learning.

Technological Features of Predicate Devices

The current and unique invention transcends other high and low magnitude output devices including, repetitive Transcranial Magnetic Stimulation(rTMS), electrical stimulation, and light and sound stimulation, which have been developed and are in existence as prior art. Each of these three types of predicate devices is addressed below. These predicate devices have been developed both for health care and personal enthusiast use. Related to health care, medically and scientifically controlled transcranial stimulation of psychiatric patients and others with psychological or psychiatric disorders has been evidenced with the development of Electroconvulsive Therapy during mid-1900s.

As related to self-care and self-improvement purposes, rTMS and other means of “magnetic therapies” have emerged during the latter part of the 20^{th} century. A low magnitude electromagnetic flux density device patented for the stated exclusive purpose of meditation enhancement (Kim, U.S. Pat. No. 6,527,696) is the most recently related innovation.

A. rTMS Predicate Devices

There have been two classes of devices patented in this area. These include the high and the low magnitude electromagnetic flux density transcranial stimulation output devices.
A. 1. The high output electromagnetic flux devices have been developed for healthcare purposes, and have primarily been driven by mental health and central nervous system health care needs. These predicate healthcare devices have focused on high output of electromagnetic flux density for a short duration. The level of electromagnetic flux density used by such devices has typically been in the 1 to 4 Tesla range. An electromagnetic flux density emitter, which is placed adjacent to the subject's scalp, delivers the electromagnetic energy field. The value of transcranial electromagnetic field delivery was discovered within the context of Magnetic Resonance Imaging (MRI), wherein patients with depressed mood who were receiving MRI procedures noticed mood elevation subsequent to the MRI procedure. This led to the development, research and use of rTMS procedures. These predicate high output devices were originally developed as an alternative to Electro-Convulsive Therapy (ECT), also known as shock therapy, in which clinically depressed patients receive a direct electrical current to the central nervous system in order to relieve symptoms associated with severe psychiatric depression, also known as Major Depressive Disorder. The high output medical rTMS devices originated and were developed to eliminate the severe and negative side effects of ECT, while promoting symptom reduction. These predicate devices use short bursts of electromagnetic flux density. The output levels of electromagnetic flux density demand skilled technicians and medical supervision. Reported side effects from such magnitudes have been investigated and found to be minimal. These devices have used single electromagnetic flux density emitter stimulation. These emitters have traditionally had a problem of overheating during stimulation. This medical procedure, while still in the experimental stages in this country, has been shown to be quite costly to consumers in other countries that have moved to the treatment phase of rTMS.
B. 2. The predicate low output pulsing electromagnetic device has been limited to one electronic device capable of delivering low electromagnetic flux density via a single low electromagnetic output emitter. This predicate electronic device designates the emission of three frequencies, 4, 8, and 10 Hz. This predicate device fixes the adjustment of output magnitude into five levels of electromagnetic flux emission. Said predicate device fixes the nature of frequency output into a pre-defined frequency ramp-down sequence that is specified to be from 20 Hz to the desired slowest frequency, and is then held static or fixed during the duration of the set program of operation. Each program mode holds a pre-designated, fixed and non-adjustable time schedule. The predicate device uses a ceramic oscillator and delivers a square wave to the emitter. The predicate device's specified electromagnetic emitter is placed around the head, similar to the placement of an athletic headband and emits approximately 135 Milligauss of electromagnetic flux.

C. Electrical Predicate Devices

Related to the current invention are Cranial Electrotherapy Stimulation devices. Such devices, when used for mental health and relaxation purposes, generally rely on stimulation frequencies of the 8-12 Hz range and remain static in their time harmonic frequencies. Furthermore, they are limited to one set of two electrically conductive electrodes, which contact the skin and are used to complete the electrical circuit through the living tissue. While these may be used as home training or therapy devices, the programming capability of said devices is functionally limited. The predicate devices have not included features designed to provide operant or classical conditioning of the central nervous system or peripheral nervous system, or other learning methods. The predicate devices have not included multiple output and/or location stimulation features or techniques, where magnitude, frequency and/or phase characteristics may influence neuronal firing and regulation of the central or peripheral nervous system. The specifically stated objectives of Cranial Electrotherapy Stimulation devices is similar to or the same as those devices delivering electromagnetic stimulation, e.g., treating anxiety, depression, and pain.

Unlike electrical stimulation, electromagnetic stimulation devices can accurately target tissue, both with regard to shape and depth of stimulation pattern. It can be highly focused and localized. Specifications of electromagnetic flux density size, shape and depth of tissue penetration can be determined with properly designed emitters. Electromagnetic flux density characteristics can be more accurately determined than can electrical stimulation, particularly when considering the central and peripheral nervous systems.

Unique to this invention are method design features, which allow for automated operant and classical conditioning and other learning methods. In addition and uniquely, this invention provides for multiple stimulation output emitters and emitter types, and allows for simultaneous multiple location stimulation. This permits complex patterns of multiple stimulation techniques including the control of such factors as frequencies, magnitudes, and timings within a complex programmed session of stimulation outputs across brain locations, for example, so as to condition and entrain tissue into more adaptive and productive functioning.

C. Sound and Light Stimulation Predicate Devices

have been existence for years and a favorite method of brainwave entrainment. The main drawback of these devices is their inability to focus stimulation, into specific and desirable areas. They use visual and auditory channels exclusively.

### DESCRIPTION

This invention consists of a portable electronic apparatus that, with its component emitter parts and systems, delivers electrical, electromagnetic and other manner of tissue stimulation, and includes and houses:

1.0 The Time Harmonic Unit (THP) provides innovative electronic features for the creation and delivery of stimulation including:
1.1 Multiple time harmonic stimulus delivery output ports, to which accompanying electromagnetic and electric delivery emitters, are attached and to which additional delivery emitters chosen by the user may be added.
1.2 Multiple, specific, variable and ordered patterns of electrical energy delivery to the specified electromagnetic, electric and any other manner of stimulus delivery emitter.
1.3 THBC (THBC) components that include embedded sound and video inputting, importing, recording and playback features activated through the THP's switching devices, and recording and playback ports.
1.4 Locking and unlocking mode and feature selection, programming and settings to insure safe, efficient and productive use outside a healthcare provider's clinical or research facility.

2.0 Multiple electric delivery emitters.

3.0 A housing unit specifically designed to house and hold in place multiple emitters for delivering complex stimulation patterns, processes and methods of stimulus delivery.

4.0 Hardware, firmware and software specifically designed for this invention that integrate and deliver multiple processes, features, functions, protocols, as well as conditioning and learning methods.

5.0 Hardware, firmware and software interfaces specifically designed for this invention that provides THP communications with a host computer or other manner of programming the THP.

This device, as well as the methods and procedures described herein, are intended for a plurality of uses in a wide variety of contexts—wherever control of internal processes disrupted by external stressors or individual susceptibilities would contribute to health and optimal performance—including, for training of focus and control in military and combat training, for maintaining alertness over prolonged periods of time, for increasing learning capabilities among the population afflicted with learning disabilities, for home use for self-improvement, self-care and self-treatment strategies, and for use within professional health care offices, clinics, hospitals and research facilities under the guidance and direction of appropriate professional personnel. This device can be used to treat a multiplicity of disorders and diagnoses within the classes of physiological, psychophysiological, psychiatric, neurological and behavioral disorders, as well as a vehicle for further research in the methods, procedures and protocols for enhancing tissue adaptive functions, including peak performance objectives.

The current apparatus includes design features to allow lay consumers, whether or not under the supervision of a health care professional, safe operation while altering their own central nervous system's, peripheral nervous system's, and musculoskeletal system's electro-chemical energy flow, within neurons and other living tissue. This invention includes intended independent use within the privacy of the consumer's own home or location of personal choice, and/or within the clinic, office, research facility, hospital or any other such facility of the health care provider or researcher. The objective of this invention is to promote self-improvement, enhancement of self-care, symptom reduction, health and healing, and peak performance characteristics of central and peripheral nervous systems, and other bodily organs and systems.

The unique qualities of this invention fall into four categories:

1. The technological features, including the unique combination of application processes;

2. The multiplicity of electromagnetic, electrical and other manner of stimulation emitters;

3. The multiplicity of processes of and techniques for: electromagnetic, electrical and other manner of stimulation delivery; and

4. The intentional use of classical, operant and other manner of learning and conditioning procedures, including customized learning and conditioning methods such as THBC (THBC).

The apparatus described in this invention has unique features that distinguish it from existing low magnitude rTMS, electrotherapy, and other manner of stimulation devices:
1. User settings control duration of session and of session components, as a means of conforming to healthcare provider and researcher needs, for promoting more effective tissue functioning.
2. User settings control frequencies and frequency sequences for the delivery of stimulation, as a means of conforming to healthcare provider and researcher needs, for promoting more effective tissue functioning.
3. User settings control magnitudes and magnitude sequences for the delivery of electromagnetic flux, as a means of conforming to healthcare provider and researcher needs, for promoting more effective tissue functioning.
4. User settings control emitter timing sequences for stimulus delivery of electromagnetic flux, as a means of conforming to healthcare provider and researcher needs for promoting more effective tissue functioning.
5. User settings control various multi-emitter timing and phase features across multiple stimulation emitters, as a means of conforming to healthcare provider and researcher needs for promoting effective tissue functioning.
6. User settings control integrated Time Harmonic Biodesensitization and Conditioning (THBC) methods and components including record and playback of sound and video output.
7. Unique password protected hardware, firmware and software interface, which connects the THP to a host computer.
8. Through the THP and host computer interface, which uses software unique to this invention, qualified healthcare practitioners and research professionals may establish, lock, and unlock any programmable settings. Such settings include sound and video input and output, multiple emitter delivery controls, frequency, phase, magnitude, and sequencing features. Unique to the software of this invention, security features are implemented, including authentication and authorization of the user, for purposes including safety enhancement. Advanced THP features will be locked out by design default for safety reasons, and unlocked and programmed only by trained and qualified health care practitioners and researchers, as a means of enhancing safety, while allowing advanced and qualified users access to the more advanced and complex therapeutic and research features.

The unique features of this invention add to the more fundamental features and types of features present in predicate devices. The current invention surpasses the fundamental features in predicate devices by including:
1. Hand-held and portable stimulation device which allows complex programming of complex stimulation features and characteristics;
2. The delivery of low magnitude electromagnetic flux densities and electrical frequencies and magnitudes, which are programmable in a complex manner, including the delivery of varying electromagnetic flux density and electrical stimuli across multiple emitters during a single stimulation session;
3. A multiplicity of user programmable modes, features, programs, and protocols, as well as design features oriented around conditioning and learning principles, sequences, procedures and protocols, including THBC.

The physical apparatus of the current invention consists of five basic components:
1. A programmable Electronic THP device, which produces an adjustable electrical current, and possesses the programming capacity for THBC and complex programming of the multiplicity of frequencies and patterns of electromagnetic flux densities, electrical and other manner of stimulus output delivery. The THP has unique features, making it ideal as a home-training device for individuals under the care of a qualified medical professional and as a medical device suitable for use by health care delivery and research professionals. Said THP includes the following characteristics and parameters:
   a) Power on/off, Mode and Start switching device;
   b) Visual displays allows the individual to view, make and confirm changes to a multiplicity of features, including those outlined in the diagram below;
   c) Illuminated displays;
   d) Illuminated switching devices;
   e) Mode selection allowing display rotation between modes at the user's discretion;
   f) Program duration selection;
   g) Output port for headphone, ear-buds or other manner of audio output device;
   h) A pair of ear-buds will be included as an adjunctive feature;
   i) Input port for digital sound and voice recording;
   j) Input port for digital video;
   k) Output port for digital video playback;
   l) Internal microphone for digital sound recording, as a means of THBC method development and execution;
   m) Interface port for connecting and interfacing the THP with a host computer or other hardware device;
   n) A multiplicity of output ports for accepting electromagnetic, electric or other manner of stimulation device emitters;
   o) Compartment which accepts standard pre-charged electrical storage and delivery unit(s), which power the THP;
   p) Illuminated battery charge status indicator;
   q) Static pulse frequency selection for output stimulation;
   r) Dynamic pulse frequency output selection. The system can be adjusted to ramp up and ramp down, or maintain constant frequency. There is from-to frequency setting capability;
   s) Static electromagnetic flux density output magnitude selection;
   t) Dynamic magnitude adjustment selection. The system can ramp up, ramp down, or maintain a constant magnitude. There is from-to magnitude setting capability;
   u) Static emitter timing adjustment capability, specifying the static timing setting between two or more emitters;
   v) Dynamic phase emitter output timing adjustment capability, specifying timing shift of the stimulus delivery, such that the outputs deliver either time harmonic stimulus closer together, or further apart, closer together and then further apart, further apart and closer together, or a combined and programmed sequence including both the closer together and then further apart and further apart and closer together, as the program proceeds through its sequence and duration, as a means of facilitating adaptive and functional tissue activity;
   w) Multiple output ports for use with one or more electromagnetic, electric, or other manner of stimulation emitters;
   x) A digital sound and voice recording, input, and playback component, as a means of developing and executing THBC methods, procedures, processes, protocols and techniques. The feature provides the capacity for customized conditioning, re-conditioning and other learning methods, processes, procedures, protocols and techniques. Said audio feature allows for addressing external environmental and internal environmental triggering stimuli, as a means of desensitizing and reconditioning undesirable central and peripheral nervous system, and musculoskeletal, and other body and brain related responses and symptoms;
2. One or more electromagnetic emitters, which are attached to the electronic THP device, through the THP device's output emitter ports. The emitters are attached to the THP by a sheathed two-conductor wire and standard matched connecting plug. Inserting the plug end of the emitter into the THP device port and initiating the Start sequence completes the electrical circuit, resulting in electromagnetic flux output through the emitter. A multiplicity of emitters may be used by the consumer, by health the care professional or researcher, for and with the consumer. The emitters vary in magnetic wire diameter or gauge, or other materials; emitter size; emitter shape; the number of times the magnet wire is wrapped onto the emitter or amount of other materials used to make said emitters vary in flux density or shape and penetration depth and shape of electromagnetic flux. The parameters for said emitters are as follows:
   a) Magnet wire diameter or gauge contributes to intended electromagnetic flux output magnitude and said wire gauge used is standardized in manufacture;
   b) Emitter size allows for localizing the flow of electromagnetic flux according to user needs;
   c) Emitter shape allows for localization of electromagnetic flux according to user needs;
   d) Emitter core type allows for determination of electromagnetic flux shape, penetration depth and flux density;
   e) The numbers of windings contained in the said emitter contributes to electromagnetic flux output magnitude;
   f) One or more cap-like Emitter Housing Units (hereafter EHU), fitting over the scalp, is capable of holding one or more emitters against the scalp and allows for consistent scalp placement and electromagnetic flux stimulation.
   g) The EHU will accommodate emitters within one or more standard placements including: the 10/20 international EEG placement system and any alternate placement system;
   h) Other materials, including ferrofluidic materials, will be used to emit, change and optimize emission characteristics of the emitters pursuant to the requirement of the individual users.
3. One or more electrically conductive electrodes of various sizes and shapes that are attached to the electronic THP device through the THP device's output stimulation ports. The electrodes are attached to the THP by a sheathed two-conductor wire and standard matched connecting plug. Inserting the plug-end of said electrode into said THP device port and initiating the “On” and “Start” sequences completes the electrical circuit, resulting in electrical output through said electrode. A multiplicity of said electrodes may be used by the consumer and by health the care professional, for and with the consumer. The electrodes vary in the nature and type of the electrode head.
4. One or more stimulation output device types and stimulation emitters, including light and sound stimulation output emitters. The emitters are attached to the THP, by a sheathed multi-conductor wire and standard matched connecting plug. Inserting the plug end of said electrode into said THP device port and initiating the “On” and “Start” sequences completes the necessary circuits, resulting in the desired and specific output through the specific emitter type, e.g., light and/or sound. A multiplicity of said emitters may be used by the consumer and by health the care professional, for and with the consumer. The emitters vary in the nature and type of the emitter used.

Unique methods and processes, allow the delivery of a multiplicity of programmed and selected output features, including:
a) electromagnetic flux;
b) electrical and other manner of stimulation;
c) use environment variation;
d) complex and programmable pulse timing variability;
e) complex and programmable magnitude variability
f) number and type of emitters used simultaneously;
g) classical, operant and other manner of conditioning, and learning methods and techniques;
h) practitioner lock-in/lock-out features and procedures;
i) sound input and output features;
j) Video input and output features.
5. Multiple Emitter Containment and Housing Apparatus Of this invention contains and holds emitters within the designated position for the purpose of adhering to the established International 10/20 EEG Electrode Placement System.

The methods and processes utilized in the current invention are unique when compared to the predicate devices in its diverse functioning capability and its adaptability.

The device is easy to use. The usage parameters maintain sufficient simplicity that accompanying directions, in either written or oral form, can be utilized directly by the consumer for immediate application. The device is sufficiently practical that the end-user consumer and the health care practitioner can program an array of included technical, method and process features.

The device incorporates principles of behavioral and psychologically oriented conditioning and learning. Where predicate devices and device types rely on relatively rigid electric, electromagnetic, light, and sound activation for entrainment purposes during the activation sessions the current invention also incorporates learning and conditioning methods, procedures, techniques and strategies that are well-known and practiced in more traditional mental health service delivery systems. This invention contains methods and processes, which may be used to transcend each training program or session toward permanent conditioning, re-conditioning and other manner of learning. The current invention possesses the capability to take advantage of a long established and well-known and scientifically documented conditioning processes such as classical conditioning (first discovered and researched by Russian Physiologist, Ivan Pavlov) and Operant Conditioning, (first established by B. F. Skinner). Learning and conditioning features and principles may be activated and augmented through the audio import and recording input, and sound and video playback features of the current invention.

The device contains lock-in features that enhance safety and focused learning. The features, methods and processes incorporated in this invention, allow the health care professional to lock in certain features, methods and processes and lock out certain features, methods and processes, enhancing the invention's public safety.

The device is portable. The device methods and processes may be operated by either the health care professional, researcher, or by the consumer end-user in physical locations they choose.

### SUMMARY

The invention provides advances in expanded forms of tissue stimulation and application methodologies for both existing electromagnetic and electric time harmonic devices in use by western medicine developed and self-improvement, consumer-based electromagnetic, electric, and light and sound time harmonic devices. This invention's diverse functional capability, its technological innovations, its programmable capacity, its adaptability and its safety and ease of use characteristics have never been provided in a single unitary product. The technological features, the emitter features, and the method and process features available in this invention, hold a multiplicity of unique elements, including:
1. A portable hand-held and consumer-friendly time harmonic device with microprocessors that allow programming and re-programming of a multiplicity of modes, features, methods and procedures;
2. Integrated THBC components including audio and video import, input and recording features, which transcend limitations of predicate devices;
3. Integrated THBC components including audio, video and digital file transfer capability;
4. Pulse timing features which have the capacity to vary in both a simple linear manner and in complex fashions within individual emitters, and have the pulse timing characteristics programmed across a multiplicity of emitters;
5. A multiplicity of pulse frequency selections allowing variable and complex frequency distributions within and across emitters and during the duration of any program;
6. A multiplicity of time harmonic magnitude selections allowing variable and complex magnitude distributions across and during the duration of any program;
7. A multiplicity of electrical, electromagnetic flux and other manner of stimulation output ports;
8. A lock-in, lock-out feature designed for consumer ease of use and safety;
9. A storage and export log feature for tracking THP use by the end user;
10. Integrated THBC method components;
11. Computer interface capable for allowing programming and re-programming of all features, programs, processes, methods and techniques in the THP.
12. A multiplicity of emitters and emitter types, including custom emitter design and manufacture, suitable for the multiplicity of stimulation uses;
13. A multiplicity of emitter housing units (EHU) for holding the multiplicity of emitters in place at various designated locations for various stimulation purposes;
14. A multiplicity of features, methods and processes designed to facilitate conditioning and re-conditioning, and other manner of learning used in various conditioning techniques and strategies.

In addition to this device having more output flexibility than the predicate home use device, this invention is uniquely capable of a lock-in, lockout safety and control feature, which may control and deny consumer programming and setting changes. This allows health care providers to lock-in and lockout certain functions from user access. Said lock-in, lockout feature enhances this invention's safety for the consumer end user. Said lock-in, lockout feature also enhances the probability of clients and patients following the methods, procedures, protocols, processes and techniques prescribed by the health care professional. No device to date has provided for these enhanced use and safety features.

This invention includes a multiplicity of selection features, including features unique to this invention. The ability of this invention to entrain neuronal modulation patterns in multiple locations and to either increase or decrease coherence and comodulation of neuronal firing patterns between brain locations has heretofore been unavailable in pulsing electromagnetic delivery systems, also known as rTMS devices.

This invention offers methods and processes, which are unique to this invention, including THBC. Components including audio and video import, input, recording and playback allow a multiplicity of methods, procedures, techniques and strategies, which may be used in a multiplicity of conditioning and learning methods, procedures, protocols, techniques and strategies. This invention is unique in bridging a gap between existing medical electromagnetic and electric pulsing devices and the self-improvement electromagnetic and electric pulsing devices, as well as adding features not present in either medical or self-improvement categories of this technology class.

### BRIEF DESCRIPTIONS OF THE DRAWINGS

FIG. 1 is a top view of the device depicting the various primary components.

FIG. 2 is a functional diagram of the apparatus which contains the main functional components including the flow between the components.

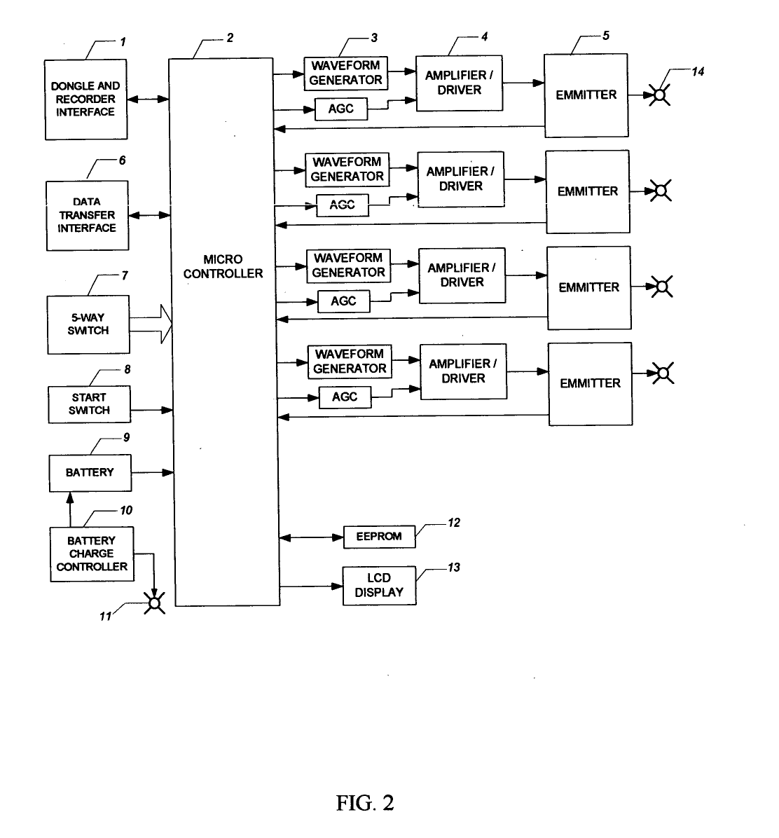

FIG. 3 shows one embodiment of an electromagnetic emitter.

FIG. 4 shows one embodiment of an electromagnetic emitter.

FIG. 5 shows one embodiment of an electric emitter.

FIG. 6 shows one embodiment of an electric emitter.

### DETAILED DESCRIPTION

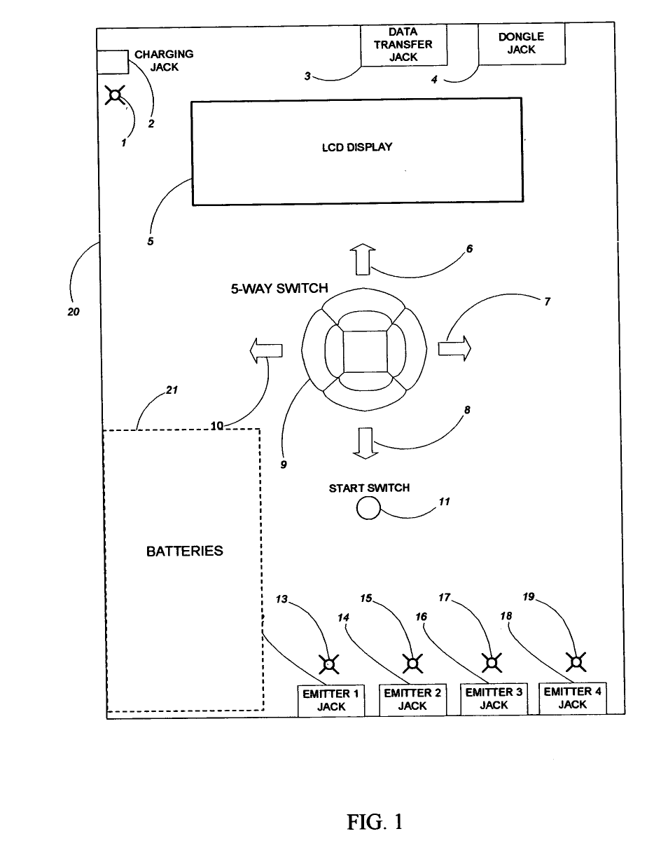

Referring to FIG. 1 the apparatus 20 is comprised of nineteen components described as follows.

An indicator LED 1 which displays charging information for the internal batteries 21 when the apparatus is being charged by an external power supply.
A power jack 2 which is used for connection for the external power supply.
An interface jack 3 used to connect the apparatus to an external computer system.
An electromechanical jack 4 used to interface to an external hardware apparatus for the purpose of authorization of the end-user, authentication of the end-user, a and recording of critical events from the apparatus. The external hardware apparatus employs an embedded power source to maintain non-volatile records and is independent of power from the apparatus. This facilitates a plurality of purposes including but not limited to patient compliance and security.
A Liquid Crystal Display (LCD) 5 that displays the various settings, modes, activities that occur over time and additional information to the user.
A five-way switch 9 that is used to perform the various tasks, settings, modes, and function of the apparatus. The five possible switch choices are up, down, left, right, and center. These selections correspond respectively to the arrow indicators 6 [up], 7 [right], 8 [down], 10 [left]. Center selection choice is achieved by pressing the center of the button.
The aforementioned arrow indicators 6, 7, 8, 10 that represent the various button selections from the five-way switch 9.
A momentary contact start switch 11 that initiates the session as programmed by the end-user.
Four emitter jacks 12, 14, 16, 18 that are used to connect to the plurality of stimulation emitters. Any stimulation emitter can be used in any of the four jacks.
Four indicator LEDs 13, 15, 17, 19 that display the emitter activity.
An internal rechargeable battery power source 21.

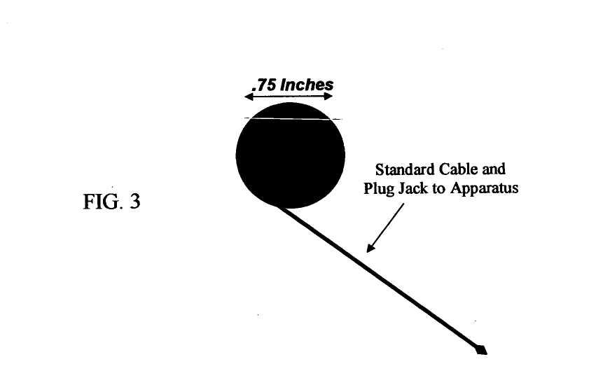

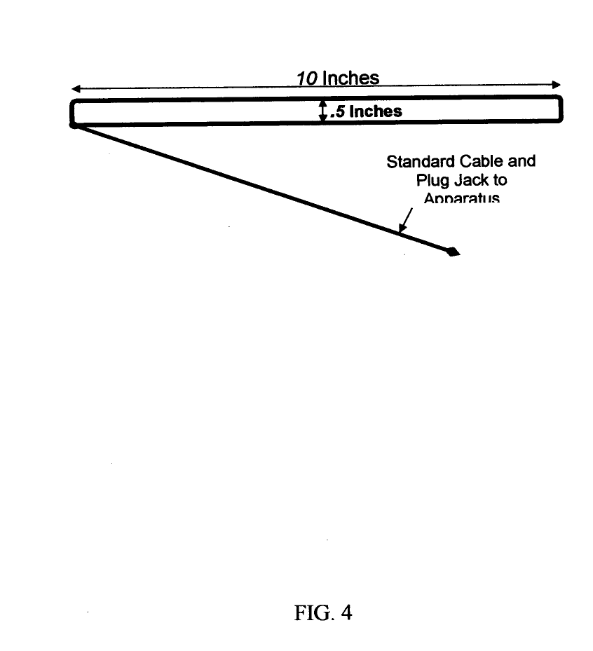

FIGS. 3 and 4 depict a sample of two embodiments of a multiplicity of electromagnetic emitter sizes, shapes styles and materials that serve as the delivery emitters of Electromagnetic stimulation. The emitter(s) are attached to the time harmonic processor via the standard plug end of the emitter. Once activated, the time harmonic processor delivers the time harmonic signal, as programmed, to the emitter port(s) and the emitter(s) are then activated and produces the electromagnetic flux, within the parameters of adjustments, settings and programming that has been entered by the user, health care professional or researcher.

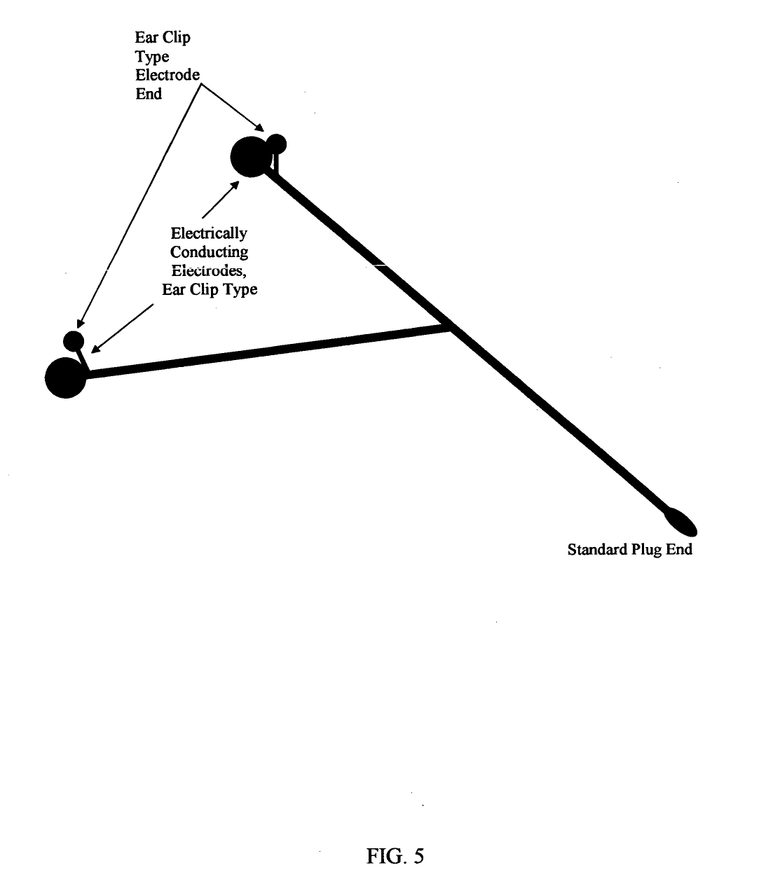

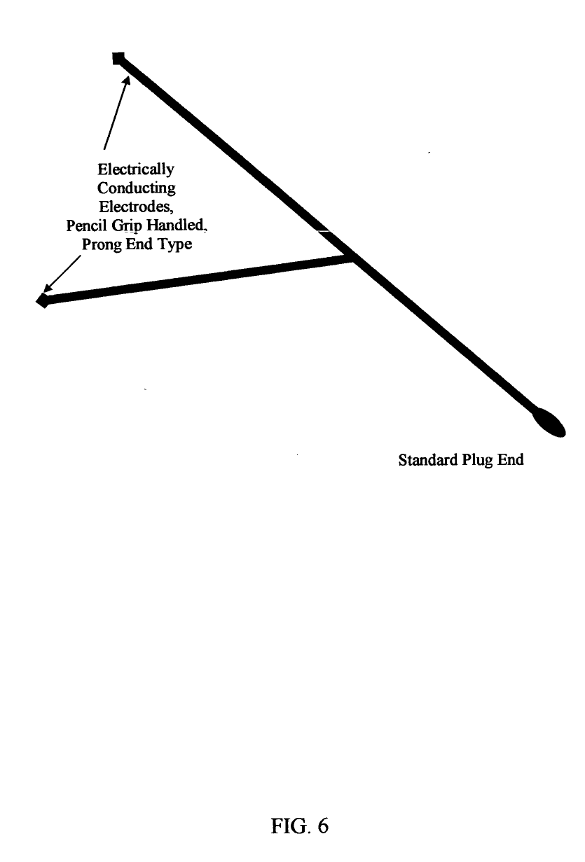

FIGS. 5 and 6 depict a sample of two embodiments of the multiplicity of shapes and styles and sizes of electrical stimulator electrodes that serve as the delivery agents for the electrical stimulator function of the time harmonic processor. Once activated, the time harmonic processor delivers the electrical time harmonic signal, as programmed, to the emitter port(s) and the electrodes are then activated and produce the designated electrical current, within the parameters of adjustments, settings and programming that has been entered by the user, health care professional, or researcher.

### THE PREFERRED EMBODIMENT WILL INCLUDE

1. THP to computer interface with both an auto-locating feature and a manual selection of port feature to be used when connecting the THP to a computer. The connection may be through a designated cable or other manner of connection.
2. Stimulation Output Ports allow the user to set the desired number of ports to be active.
3. Frequency mode allows the user to determine and select the specific frequency of electric pulse to be emitted, including: static, dynamic and phase characteristics.
4. Magnitude mode allows the user to determine and select the specific magnitude of electric pulse to be emitted, including: static, dynamic and phase characteristics.
5. Timing mode allows the user to determine whether a programmed session will include static, dynamic and phase characteristics.
6. Duration mode allows the user to determine the duration of a programmed session;
7. Audio allows the user to determine the audio characteristics to be present in a programmed stimulation session, including turning the Audio mode on and off, recording, importing and exporting of audio data.
   Operation

One of the unique benefits of the current invention is its ease of operation and portability for the consumer, while maintaining a multiplicity of features of interest to the qualified health care professional. The consumer, whether a lay consciousness enthusiast, or a patient suffering from any number of maladies, may purchase this invention and use it in its default safe modes. When desirable and necessary the consumer may be instructed by a health care professional, either in an office or remotely. The qualified health professional will have full access to the THP through password-protected software. The professional's training will allow for the adjusting of modes and settings in said THP programmed features and settings.

Consumer Operation

Consumer operation or this invention contains numerous steps, which will be outlined in this section. Some of the operational steps are specific to particular applications or objectives (including THBC methods and procedures), as well as to localization of Electromagnetic flux, electrical, or other manner of energy output, and as related to particular central, peripheral nervous system, musculoskeletal change, symptom reduction and/or enhancement of bodily functions. Some of the operational steps are uniform across applications and methods, while others are specific to particular applications. More than one emitter type may be used simultaneously. They will be described within these distinctions.

Electromagnetic emitter selection and placement localization will be outlined as specific examples. The consumer or practitioner first selects the emitter type, e.g., electromagnetic flux emitter, consistent with the particular objective(s). Following emitter type selection, the emitter or output device must be placed against or adjacent to the scalp or other bodily location.

The following examples will illustrate two of a multiplicity and varied placement and localization characteristics and possibilities available in, and as a part of, this invention. While these examples each represent a specific symptom set or disorder to be treated, these are only examples of a multiplicity of symptoms, conditions, and disorders that may be treated and that are available within this invention. While these examples address electromagnetic stimulation, other output devices may be substituted, with other specific stimulation approaches being used. The two examples are as follows:
1. Example one. A emitter that is flat on the top and bottom and round with, for example, a one inch diameter, may be used for localizing electromagnetic stimulation, in order to reduce psychological or psychiatric depression symptoms. The consumer or health practitioner places the Spot Emitter at a predetermined location. In this example, said Spot Emitter is placed or localized at the left temple area of the individual's head, a location known as F7 in the International EEG 10/20 electrode placement system. Said Spot Emitter may be affixed with this invention's emitter housing unit (EHU), which houses these 10/20 locations and the method for affixing the emitter appropriately. Likewise, a hair band or any other suitable device may be used to locate and hold in place the emitter. The apparatus of this invention is then activated according to the desired output characteristics. For example, the time harmonic stimulation delivery sequence might be set to emit a frequency of 14 Hz at the magnitude of one Gauss, and for the duration of one hour.
2. Example two. A U-shaped Emitter may be selected for the elimination of chronic pain. To illustrate this example, being one of a multiplicity of placements for a multiplicity of conditions and symptoms that would use the U-Emitter, the individual may place the U-Emitter over the top of the head, with each end of this elongated emitter resting above the ears. This emitter, in this particular example of localization, would span from the left to right side over the top of the head and would be centered from side to side and “pointing” to the ears. This U-Emitter would rest across the top of the head and said emitter placement would correspond to and rest on the International EEG 10/20 electrode placement system locations designated as C3, Cz and C4. This area is also corresponds to the sensory motor strip of the brain. Said U-Emitter would be held in place by this invention's EHU or by any other manner of device that affixes the U-Emitter in place. The apparatus of this invention is then activated according to the desired output characteristics. For example, the rTMS sequence might be set to emit a frequency of 12 Hz at the magnitude of one and one half Gauss for the duration of forty five minutes.

The consumer, in either of these two examples, or in any of the multiplicity of placements for the multiplicity of symptoms or conditions of the central and peripheral nervous systems, and for any of the multiplicity of symptoms or conditions of the musculoskeletal system, may then plug the opposite end of the emitter into the THP. In the above described single-emitter examples and use method, the user plugs the THP plug end of the cable attached to the emitter into output port labeled number one (EP 1). In this described use of electromagnetic output port number one has been set as the default designation for the THP, when using one electromagnetic output emitter and method.

Continuing with the examples above and expanding the detail of operation, after the consumer or practitioner has powered on the THP using the Power/Mode/Start switching device or other power on switching device as may be present, next initiates the mode selection sequence with the switching device.

The user may then select and observe the THP mode function or operation by viewing the relevant Display. With the THP powered on and in the Mode position, any desired changes may be made by the user.

If a health care professional has pre-programmed a sequence or complex sequence of events and patterns of stimulation and has initiated the lock out feature, the end-user will next press the selector to begin the sequence.

In the event that the user has programming characteristics at his discretion, or that a health care professional is programming the device, the user next selects the desired mode through the switching device, which is viewed by observing the Mode Display. For example, the user may select frequency, magnitude, timings, duration, THBC, including various other modes that are present for use. Said switching device, when depressed on the right side, or through other instructed manipulation, moves forward from mode to mode and when depressed on the left side, or through other instructed manipulation, moves backward from mode to mode.

The mode-switching device allows the user to select any mode and in so doing, allows for parameter adjustment or setting of any selected mode. The selected and active mode is viewed through the mode display window. This will be illustrated through an example of the Mode and the “Setting” manipulation, on one of the multiplicity of modes that may be selected. For example, the user may desire to change the frequency of electronic time harmonic and set it to a specific frequency.

Once the frequency mode is selected and active, the user selects the desired frequency adjustment or setting by manipulating the appropriate switching device. In this example, the user finds that the THP has been previously set to provide 10 Hz stimulation by observing the “Setting” display, while in Frequency mode. The user may manipulate the switching device to reset the THP to the time harmonic of 15 Hz, for example. The user may then reset to maintain a 15 Hz time harmonic pattern throughout the program or session. Selecting and adjusting the desired frequency of THP time harmonic, the user may increase or decrease THP time harmonic frequency.

The selected and active frequency setting is viewed through the “Setting” Display Window. The user then manipulates the Mode switching device to make active and display the next desired mode for the “Setting” adjustment. For example, the user may desire a decrease or increase in magnitude of stimulus delivery. The user manipulates the switching device to display the electromagnetic flux magnitude mode.

The user then views the “Setting” display and observes the current setting for stimulation magnitude output. Using the “Setting” switching device the user then may change the stimulation magnitude output from the THP. The user may then again manipulate the Mode switching device. The user may then select the Duration mode for adjustment, by using the Mode switching device again. Once the Duration feature or mode has been selected, the user will use the “Setting” switching device to adjust to the desired length of stimulation session. In each case, the user confirms the selected setting by switching to the next mode or by starting the system through manipulating the appropriate switching device, which is so indicated and viewable through the Display system. In following these steps, the user has control over the frequency, magnitude, and duration, as well as various other modes and settings related to the particular stimulation delivered, once the system is started.

The THP may then be activated and starts the stimulation and programmed phase through manipulating the appropriate switching device. Once started the display registers such activity. The programmed characteristics, including duration, frequency and magnitude of stimulation session, will proceed until the assigned stimulation duration has elapsed. At that time, the THP will shut down and stop all stimulation. Likewise, this will be true for any and all other modes engaged and settings engaged by either the consumer or health care professional. In the Activity Log Module, the THP records and logs activities, features, sessions, and settings that have been engaged by the user. This tracking mechanism is available for progress and research tracking purposes.

Health Care Professional and Researcher Operation

Qualified health care professionals and researchers will have all the capabilities of operation that are accessible to the consumer and listed above in the Consumer Operation section. Qualified professionals will also have access to and control of all lock-in/lock-out features, modes, settings and adjustments. Qualified professionals may unlock various features, modes, settings and adjustments for the patient or other end user. The end user, at will, may then manipulate any unlocked features, modes, settings and adjustments. The professional may lock features, modes, settings and adjustments from consumer access, as desired, thus maintaining exclusive control of all features, modes, settings and adjustments within the THP. In so doing, the professional considers client or patient safety, as well as ease of use for the selected therapy protocol, modality, learning, conditioning and any and all other factors that may be in the best interest of the patient or client.

While the professional may view modes features, settings and adjustments of the THP front panel, as the client or patient does, the professional may also access the THP through a computer connected to the THP via hardware and software of this invention. In so doing, the professional may make changes to: modes, features, settings and adjustments. The professional may then accept THP changes of any modes, features, settings and adjustments through the computer to THP software feature.

The professional may then lock any and all modes, features, settings and adjustments. THP interface software will be installed on the professional's computer. The THP will be connected to the professional's computer and THP software by a standard computer interface port or any other connection device supported by or designed for the THP and the computer. One end of the designated cable interface is plugged into the THP at its Computer Interface Port. The other end of said cable is plugged in and connected to the professional's computer.

The hardware connection and THP software also allow the professional to turn on and off the requirement for the entry of a designated name and password in order for the consumer to have access to any and all features, modes, settings and adjustments in and on the THP. From the professional's computer the THP software will be entered using the THP software executable file. The professional then has access to all modes, features, settings and adjustments available within the THP. The professional will, through the THP software, be able to view and make changes to all features, modes, settings and adjustments available within said THP. After unlocking any desired features, modes, settings and adjustments, the qualified professional may select all desired modes, one at a time, and make desired changes. The professional may then lock any and all features though the THP software, modes, settings and adjustments of the THP. The professional also has the capability to unlock and leave unlocked all features, modes, settings and adjustments, as desired.

As a result, and at the minimum, the consumer may have all parameters preset by said professional and only have access to the Power and Start switching devices. On the contrary, the consumer, if so desired by the professional, may be allowed access to any or all features, modes, adjustments and settings available through the THP. The default design has certain features and settings locked out in order to provide safety for the lay user and unqualified professional.

We claim:

1. A multifunction electronic tissue stimulation apparatus with integrated software application methods comprising:
1. A portable electronic device for tissue stimulation using high precision time harmonic signal generating and processing, the improvement wherein offers an expanded and programmable spectrum of tissue stimulation including electromagnetic stimulation and a plurality of other forms of tissue stimulation available in a single portable apparatus with safety features for the end-user and expandable forms of tissue stimulation delivery, comprising:
a) a device with configurable parameters, including waveform symmetry, frequency and amplitude, and
b) a device with channels for electric, electromagnetic and other manner of tissue stimulation, and
c) tissue stimulation output ports with the means for simultaneous operation, and
d) tissue stimulation emitters for the conversion of electricity to the provided forms of tissue stimulation, including non-focused, wide flux pattern and focused narrow flux pattern electromagnetic emitters, and
e) tissue stimulation emitter safety features including temperature, magnetic and optical sensors placed at the egress point of emission, and
f) exchangeable modules and PCP boards for system feature upgrades, and
g) an external dongle that regulates and bridges device data, program initiation and duration, provides session data storage, and prevents unauthorized access to the device, and
h) communication port for the transmission of data between the device and a computer, and
i) A compact, ergonomically designed case housing of the tissue stimulation device that provides for portability and ease of use in research, clinical, and home use settings.

2. Operating systems and software programs for output adjustment and control of the portable precision time harmonic signal processing and generating apparatus as claimed in claim 1, comprising:
a) automated sequential and random activation and deactivation, and
b) differential phase programming for a plurality of tissue stimulation emitters that generates complex inter-emitter phase sequences for tissue stimulation, and
c) programmable control for the selection and setting of the preferred tissue stimulation session characteristics, including pre-established default tissue stimulation sessions, and
d) programmable control of tissue stimulation session data storage, session characteristic adjustment and retrieval integration between the device claimed in claim 1 and a computer system.

3. A time harmonic biodesensitization and conditioning method contained in the device software for programming of a series of tissue stimulation sessions specifically for the operation of the device of claims 2, as a means for the improvement of adaptive tissue response to stressors, comprising:
a) tissue stimulation programs that use existing models of classical, operant, and other manner of conditioned learning in the delivery of tissue stimulation sessions in a gradated or hierarchal form of increasing levels of stimulation, and
b) methods for the entrainment of the relaxation response in the end user during the tissue stimulation sessions, and
c) methods for the monitoring and regulation of the increasing levels of stimulation with simultaneous monitoring and regulation of the end-user's relaxation response, and
d) methods for recalibrating the hierarchal stimulation levels with the end-user's capacity for maintenance of the relaxation response, in the use of the apparatus.

4. Specific protocols adapted for the application of the methodology by the end user or health care practitioner for time harmonic biodesensitization and conditioning claimed in claim 3, comprising a means for programming of time harmonic biodesensitization and conditioning methods:
a) an accompanying instructional book and audiotape for the end user with detailed descriptions of the methodology for time harmonic biodesensitization and conditioning, and
b) specific written forms and worksheets for the end user to develop personalized data for use with the apparatus, and
c) detailed descriptions of how to input the personalized data for use with the tissue stimulation sessions delivered by the apparatus, and
d) criteria and descriptions for the creation and input of time-controlled sequences of audio and video recordings for time harmonic biodesensitization and conditioning for use with the tissue stimulation sessions delivered by the apparatus, and
e) criteria and protocols for the increased development of healthy stress adaptive responses for the end user, for use with the tissue stimulation sessions delivered by the apparatus.
